(19) United States
(12) Patent Application Publication
(10) Pub. No.: US 2024/0003922 A1
(43) Pub. Date: Jan. 4, 2024

(54) POLYMERIC GEL COMPOSITIONS FOR DETECTION OF THC

(71) Applicant: XEROX CORPORATION, Norwalk, CT (US)

(72) Inventors: Gabriel Iftime, San Francisco, CA (US); Fatemeh Ostadhossein, Palo Alto, CA (US); Sean Emerson Doris, San Francisco, CA (US); Stephen Matthew Meckler, Boulder, CO (US); Mahati Chintapalli, San Francisco, CA (US)

(21) Appl. No.: 18/199,656

(22) Filed: May 19, 2023

Related U.S. Application Data
(60) Provisional application No. 63/344,402, filed on May 20, 2022.

(51) Int. Cl. G01N 33/94; G01N 33/52

(52) U.S. Cl. CPC G01N 33/948; G01N 33/521

(57) ABSTRACT

A polymeric gel incorporating a diazonium salt for detection of THC. The diazonium salt may be covalently attached to the polymer, or non-covalently distributed throughout the polymer. The polymeric gel may be formulated as a spot on a test strip to be used for drug testing. The drug may include THC. Methods of preparing a polymerizable diazonium salt precursor, called an amino aryloxyl monomer, as well as methods of preparing a polymeric gel incorporating a diazonium salt.

### CROSS-REFERENCE TO RELATED APPLICATIONS

This application claims priority to U.S. Provisional Patent Application Ser. No. 63/344,402, filed May 20, 2022, the disclosure of which is incorporated herein by reference.

### FIELD

The present disclosure relates to polymeric gel compositions for THC detection and methods for making the same. The present disclosure further relates to polymeric gel compositions prepared using diazonium salts and preparation of unique monomers for incorporation in those gels. The present disclosure also relates to devices containing such polymeric gel compositions and to methods using the devices.

### SUMMARY

The present disclosure is directed to a composition for detecting THC comprising polymerized monomers and a diazonium salt, (i) wherein the diazonium salt is covalently attached to the polymerized monomers; or (ii) wherein the diazonium salt is non-covalently dispersed throughout the polymerized monomers.

In some embodiments, the composition may include more than one type of polymerized monomer. In some embodiments, the polymerized monomers include mono-, di-, or multi-functional monomers comprising vinyl monomers, acrylate monomers, methacrylate monomers, or a combination thereof.

In some embodiments, the diazonium salt is stabilized by addition of an alternate counterion. In some of these embodiments, the counterion is thiocyanate, picrate, carboxylate, tosylate, or o-benzenedisulfonimide.

In compositions wherein the diazonium salt is non-covalently dispersed throughout the polymerized monomers, wherein the diazonium salt comprises a compound of Formula (I):

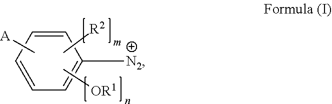

wherein A is an aromatic or nitrogen-containing group, or wherein A is absent, wherein each R¹ is independently alkyl, ether, alcohol, or aromatic, wherein n is 1 or 2, wherein each R² is independently —H, -halogen, or alkyl, and wherein m is 2 or 3.

In compositions wherein the diazonium salt is non-covalently dispersed throughout the polymerized monomers, the diazonium salt may be represented by compound of Formula (IB):

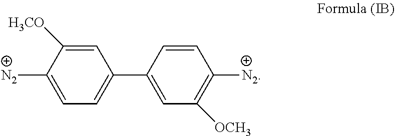

In another aspect, this disclosure describes an amino aryloxyl monomer comprising a polymerizable double bond. In some embodiments, the amino aryloxyl monomer is represented by a compound of Formula (II):

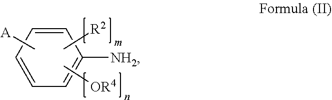

wherein is an aromatic or nitrogen-containing group, or wherein A is absent, wherein each R⁴ is independently selected from alkyl, -vinyl, acrylate, or methacrylate, wherein n is 1 or 2, wherein each R² is independently —H, -halogen, or alkyl, and wherein m is 2 or 3.

In some embodiments, the amino aryloxyl monomer is represented by of Formula (IID) or Formula (IIE):

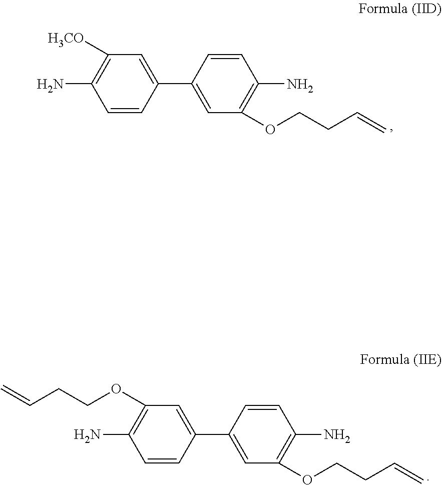

In some embodiments, in the composition wherein the diazonium salt is covalently attached to the polymerized monomers, the diazonium salt may be at least one of the molecules represented by Formula II, wherein R⁴ is attached to the polymer and wherein the amino group has been replaced with a diazonium group.

In another aspect, this disclosure describes a test device (e.g., a test strip) comprising a substantially transparent substrate and one or more test spots made of the composition for detecting THC described above. The test spots may be attached onto a porous support material, wherein the porous support material has at least one exposed surface constructed to absorb a fluid.

In some embodiments, the test spot is made of a composition wherein the diazonium salt is covalently attached to the polymerized monomers. In some of these embodiments, the diazonium salt shows less leaching during testing than when the diazonium salt is non-covalently dispersed throughout the polymerized monomers. In some of these embodiments, the diazonium salt has greater thermal stability than when the diazonium salt is non-covalently dispersed throughout the polymerized monomers.

In some embodiments, the fluid absorbed by the test device (e.g., a test strip) may be a sample dissolved in a solvent, wherein optionally the sample is at least one of blood, urine, plasma, saliva, or a combination thereof. In some of these embodiments, the solvent is a buffer with a pH of 7 or higher. The solvent may alternately be organic. The solvent may alternately be chloroform, acetone, dichloromethane, dimethylformamide, ethanol, ethylene glycol, diethylene glycol, acetonitrile or methanol.

In another aspect, this disclosure describes a method for making a polymeric composition to detect THC, the method consisting of preparing a reaction mixture including aromatic amines, polymerizable moieties, and solvent and polymerizing the reaction mixture by addition of a radical initiator to produce a polymer with the aromatic amines dispersed within the polymer. In a second step, the method includes reacting the aromatic amines to yield a composition comprising a diazonium salt and polymerized monomers.

In some embodiments, the reaction mixture includes one or more polymerizable monomers and one or more aromatic amines. In some embodiments, the polymerizable monomers comprise mono-, di-, or multi-functional monomers comprising vinyl monomers, acrylate monomers, methacrylate monomers, or a combination thereof.

In some embodiments, the reaction mixture includes one or more molecules described by Figure II.

In some embodiments, the diazonium salt is stabilized by addition of an alternate counterion. In some of these embodiments, the counterion is thiocyanate, picrate, carboxylate, tosylate, or o-benzenedisulfonimide.

In some embodiments, the aromatic amines are activated by addition of sodium nitrite (NaNO₂) and hydrochloric acid (HCl).

In another aspect, this disclosure describes a method for making the amino aryloxyl monomer described by Formula II, the method consisting of reacting an aromatic amino phenol and monomer precursor in a solvent to yield an amino aryloxyl monomer. In some of these embodiments, the monomer precursor is selected from an alkenyl halide, acryloyl chloride, or methacryloyl chloride.

### BRIEF DESCRIPTION OF FIGURES

The patent or application file contains at least one photograph executed in color. Copies of this patent or patent application publication with color photographs will be provided by the Office upon request and payment of the necessary fee.

FIG. 1 is a representation of incorporation of a diazonium salt into a polymer formed by two monomers.

FIG. 2 (1) shows reaction of a with a monomer precursor to form an aryloxyl amine comprising two polymerizable moieties. (2) shows polymerization of the amino aryloxyl monomer with additional diene monomers. (3) shows reaction of the polymerized aryloxyl amine with sodium nitrite and p-Toluenesulfonic acid to yield a diazonium group.

FIG. 3 is a 1H-NMR spectrum of divinyl compound, showing a symmetrical structure accounting for all hydrogen atoms and a pure compound.

FIG. 4 is a 13C-NMR spectrum of divinyl compound, shows a symmetrical structure and accounts for all required carbon atoms.

FIG. 5A shows photographs of a polymer with covalently attached diazonium moieties and the same polymer in solvent.

FIG. 5B shows photographs of a polymer with disperse, non-covalently attached diazonium moieties and the same polymer in solvent.

FIG. 6 shows photographs showing color reaction of a diazonium-containing polymer with THC in basic conditions to produce a color change from yellow to red.

FIG. 7 shows the absorbance spectrum of a diazonium-containing polymer incubated with either MeOH or THC in MeOH.

FIG. 8A is a diagram of a test device (e.g., a test strip) according to an example embodiment.

FIG. 8B is a cross section of the test strip of FIG. 8A according to an example embodiment.

FIG. 9 is a diagram showing drug detection using a test device (e.g., a test strip) according to example embodiments.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer”, “polymerized monomers”, and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The term “aromatic ring” is used in this disclosure to refer to a conjugated ring system of an organic compound. Aromatic rings may include carbon atoms only, or may include one or more heteroatoms, such as oxygen, nitrogen, or sulfur.

The term “alkylated” is used in this disclosure to describe compounds that are reacted to replace a hydrogen atom or a negative charge of the compound with an alkyl group, such that the alkyl group is covalently bonded to the compound.

The term “alkyl” is used in this disclosure to describe a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 10 carbon atoms, 1 to 6 carbon atoms, 1 to 4 carbon atoms, or 1 to 3 carbon atoms. Examples of alkyl groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, etc.

The term “amino aryloxyl” is used in this disclosure to describe an aryloxyl molecule that includes an amino (—NH₂) group. An aryloxyl molecule contains an oxygen molecule attached to an alkyl group and an aromatic group.

The term “substantially” as used here has the same meaning as “significantly,” and can be understood to modify the term that follows by at least about 90%, at least about 95%, or at least about 98%.

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

The term “about” is used here in conjunction with numeric values to include normal variations in measurements as expected by persons skilled in the art, and is understood to have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to polymeric gel compositions for THC detection and methods for making the same. The present disclosure further relates to sensing gel compositions prepared using diazonium salts and preparation of unique monomers for incorporation in those gels. The present disclosure also relates to devices containing such sensing gel compositions and to methods using the devices.

Legalization of marijuana for recreational purposes presents the opportunity for new public safety challenges, including tetrahydrocannabinol (THC)-impaired driving. Rapid and low-cost THC detection is needed for screening drivers, employees, and other personnel. There is a particular need for random roadside testing of drivers by police and for workplace drug testing. Current THC detection methods include immunoassays, colorimetric test kits, and confirmatory laboratory tests. Immunoassays have a relatively high cost and long processing time, making them non-optimal for high-throughput police use. Colorimetric test kits are often used in workplace testing, but consist of multiple flasks with hazardous liquids, require specialized knowledge to use, and have slow processing times. Confirmatory laboratory tests such as mass spectrometry are accurate, but not feasible for routine field use due to high cost and long turnaround times.

Ideally, police would be able to use a small, inexpensive rapid device to accurately test for THC in a suspect's bodily fluid, enabling high-throughput screening of drivers and other persons of interest. While multiple THC-sensing molecules exist, many are challenging to incorporate into commercial test assays. Diazonium salts are a class of THC-sensing molecules. Diazonium salts are very reactive and thermally unstable, making them challenging to incorporate into test spots. Additionally, diazonium salts that have been deposited on a test strip are likely to be moved by solvent during testing, resulting in poor test accuracy and reproducibility. This disclosure describes polymeric gel compositions for the detection of THC, and methods of making the same.

A polymeric gel composition for detection of THC is sometimes referred to as a sensing gel, or a THC sensing gel. A gel is understood to mean a semisolid network of polymer containing a solvent. The solvent may be water, an organic solvent, or a mixture thereof. In some embodiments, the sensing gels are prepared by polymerizing a mixture of monomers and a diazonium salt precursor, and subsequent diazotization of the precursor to yield the THC-sensing diazonium salt. Diazonium salt precursors are sometimes referred to as aryloxyl amines. According to an embodiment, test spots may be prepared from the sensing gel. Testing devices incorporating the sensing gel may provide a suitably rapid and inexpensive method for high-throughput testing. Such testing devices may be used, for example, in a law enforcement setting, in a healthcare setting, at schools and other educational institutions, at the workplace, etc.

The present disclosure relates to incorporation of one or more aryloxyl amines in a vinyl or acrylate-based polymer and subsequent diazotization of the aryloxyl amines to form diazonium groups. The aryloxyl amines may be covalently linked to the polymer by first synthesizing an amino aryloxyl monomer, which can then be copolymerized with other monomers. The amino aryloxyl group may be further activated after polymerization to form a diazonium group. Covalent attachment of the diazonium group to the polymer may result in desirable properties of the sensing gel, such as reduced leaching during testing with a solvent and improved stability. The present disclosure further relates to test strips designed to incorporate the sensing gel.

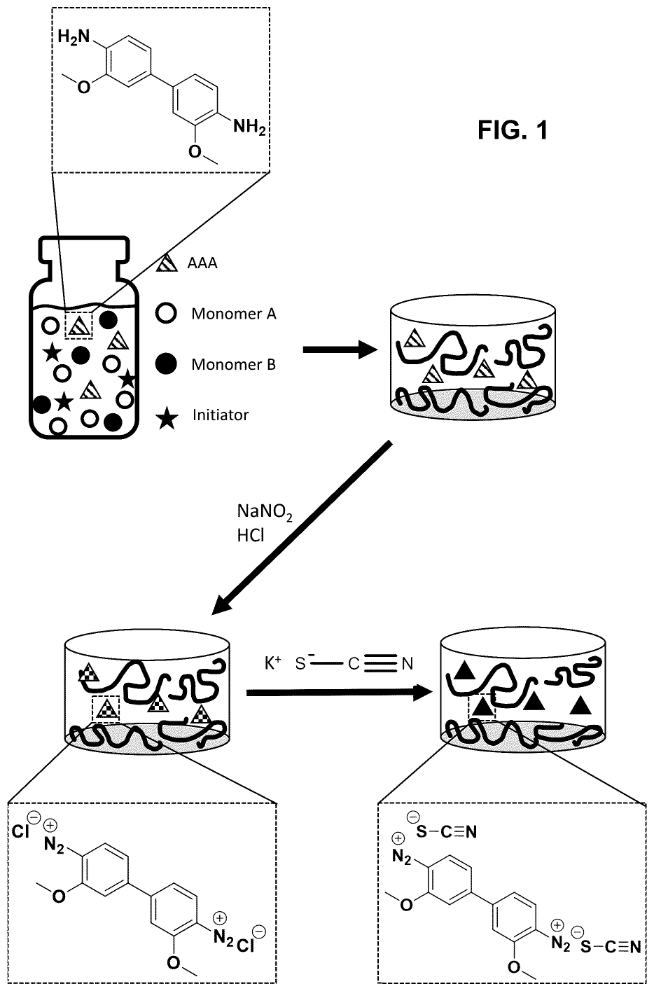

According to an embodiment, a sensing gel for detection of THC includes polymerized monomers and a diazonium salt. In some of these embodiments, the polymerized monomers in the gel may be covalently attached to a diazonium salt. In some other embodiments, the diazonium salt may not be covalently attached to the polymerized monomers, but rather be dispersed throughout the polymer, as shown in FIG. 1. The polymerized monomers may be all of the same type, or there may be a mixture of multiple types of monomers present in the polymer. In some embodiments, the monomers may be mono-, di-, or multi-functional monomers. The monomers may include vinyl monomers, acrylate monomers, or methacrylate monomers. In some embodiments, the pore size of the polymer gel may be between 5 nm and 1000 nm.

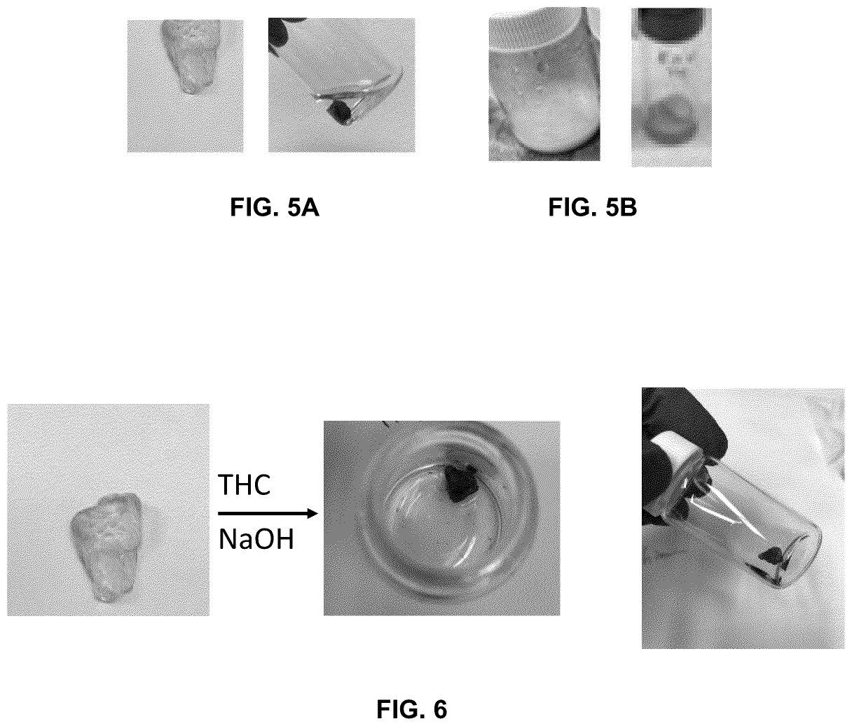

In some embodiments, the diazonium salt is covalently attached to the polymer network. Covalent attachment of the diazonium salt to the polymer network may provide certain beneficial properties. One potential beneficial property includes reduced leaching of the diazonium salt when a solvent is applied to the polymer. Diazonium salts that are covalently attached to the polymer network may not become dissolved and/or displaced when a solvent is applied. This is shown, for example, in FIG. 5A, compared with FIG. 5B, where the diazonium salt is dispersed within the polymeric gel. This may prevent loss of the salt from the polymer and maintain distribution of the diazonium salt throughout the polymer network. In some embodiments, covalent attachment of the diazonium salt to the polymer network may make distribution of the salt throughout the polymer more uniform. Another potential beneficial property of covalent attachment of the diazonium salt throughout the polymer is improved stability of the diazonium salt over time.

According to an embodiment, the diazonium salt of the sensing gel may be stabilized by a large counterion. In some embodiments, the diazonium salt may have originally been synthesized with a halogen counterion (e.g., Cl⁻). However, this counterion may have been exchanged for a larger counterion for the purpose of stabilizing the diazonium salt. Potential stabilizing counterions include thiocyanate, picrate, carboxylate, tosylate, and o-benzenedisulfonimide.

In some embodiments, the diazonium salt is not covalently bound to the polymer of the gel but is simply dispersed within the gel. The diazonium salts suitable for use with disperse, non-covalent incorporation into the sensing gel may include those represented by Formula (I):

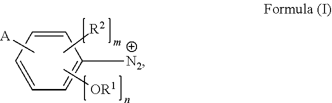

wherein A is an aromatic or nitrogen-containing group, or A is absent, wherein each R¹ is independently alkyl, ether, alcohol, or aromatic, wherein n is 1 or 2, wherein each R² is independently —H, -halogen, or alkyl, and wherein m is 2 or 3.

Alternatively, A may be NO₂, a phenyl group attached via nitrogen, or a disubstituted phenyl group. In some embodiments, A may be represented by the following structures:

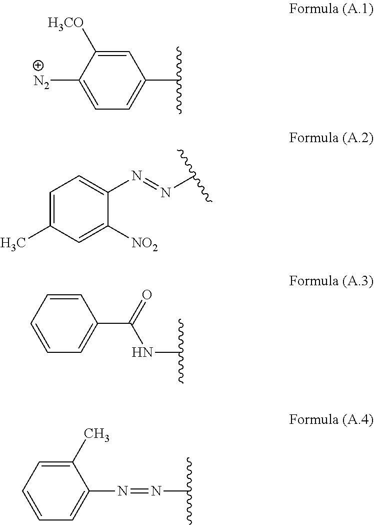

The diazonium salts suitable for use with disperse, non-covalent incorporation into the sensing gel may include those represented by Formula (IA):

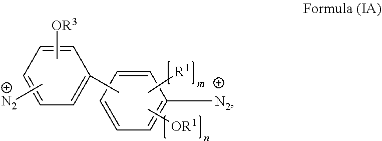

wherein each R¹ is independently H— or alkyl, wherein n is 1 or 2, wherein each R² is independently —H, -halogen, or alkyl, wherein m is 2 or 3, and wherein each R³ is independently —H, alkyl, or ether.

In Formula (I) or Formula (IA), the diazonium group and the A group may be in a para position, an ortho position, or a meta position with respect to one another.

The diazonium salts suitable for use with disperse, non-covalent incorporation into the sensing gel may also include those represented by Formula (IB):

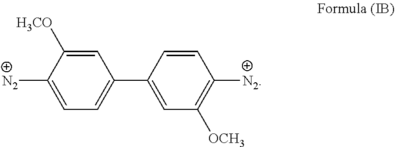

The diazonium salts suitable for use with disperse, non-covalent incorporation into the sensing gel may include Fast Blue B salt (o-dianisidine bis(diazotized) zinc double salt), Fast Blue BB salt hemi(zinc chloride) (4-benzoylamino-2,5-diethoxybenzenediazonium chloride hemi(zinc chloride) salt), Fast Corinth V salt (2-methoxy-5-methyl-4-(4-methyl-2-nitro phenyl)-azobenzene-diazonium), Fast Red B salt (2-methoxy-4-nitro benzenediazonium salt), Fast Bordeaux GP salt (4-methoxy-2-nitro benzenediazonium chloride hemi(zinc chloride) salt), Fast Blue RR salt (4-benzoylamino-2,5-dimethoxybenzenediazonium chloride hemi(zinc chloride) salt), Fast Garnet GBC sulfate salt (4,2-methyl-4-([2-methylphenyl]azo)benzenediazonium salt), or Fast Red Violet LB salt (5-chloro-4-benzamido-2-methylbenzenediazonium chloride hemi(zinc chloride) salt).

This disclosure additionally describes an amino aryloxyl monomer and methods of making the same. Commercially available diazonium salts and diazonium salt precursors do not contain polymerizable moieties, and therefore cannot be polymerized. According to an embodiment, a polymerizable diazonium salt precursor, sometimes referred to as an amino aryloxyl monomer, includes an amino aryloxyl group and a polymerizable moiety.

According to an embodiment, the amino aryloxyl monomer may be represented by Formula (II):

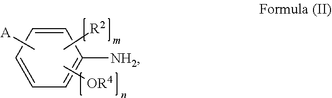

wherein A is an aromatic or nitrogen-containing group, or wherein A is absent, wherein each R⁴ is independently selected from alkyl, vinyl, acrylate, or methacrylate, wherein n is 1 or 2, wherein each R² is independently —H, -halogen, or alkyl, and wherein m is 2 or 3.

In some embodiments, the amino aryloxyl monomer may be represented by Formula (IIA):

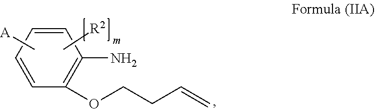

wherein A is an aromatic or nitrogen-containing group, or wherein A is absent, wherein each R² is independently —H, -halogen, or alkyl, and wherein m is 2 or 3.

In some embodiments, the amino aryloxyl monomer may be represented by Formulas IIA.1 or IIA.2:

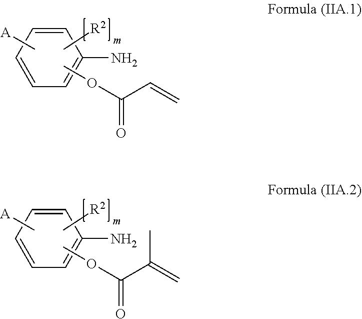

In some embodiments, A is NO₂, a phenyl group attached via nitrogen, or a disubstituted phenyl group. In some embodiments, A may be represented by Formulas (A.1-A.4) shown above.

In some embodiments, the amino aryloxyl monomer may be represented by Formula (IIB):

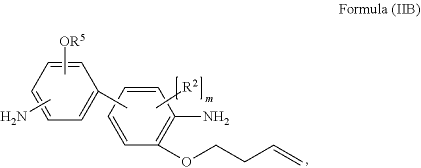

wherein each R5 is independently selected from, alkyl, vinyl, acrylate, or methacrylate, wherein each R2 is independently —H, -halogen, or alkyl, and wherein m is 2 or 3.

In some embodiments, the amino aryloxyl monomer may be represented by Formula (IIC):

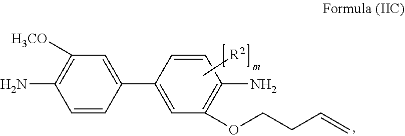

wherein each R2 is independently —H, -halogen, or alkyl, and wherein m is 2 or 3.

In some embodiments, the amino aryloxyl monomer may be represented by Formula (IID) or Formula (IIE):

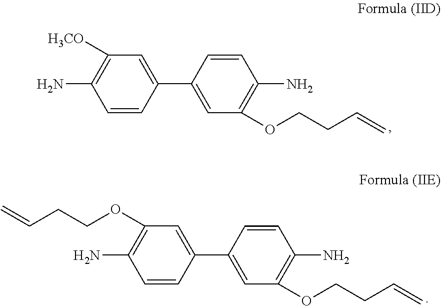

In another embodiment, a test strip may include a substantially transparent substrate and one or more test spots including the THC sensing gel. The THC sensing gel may be attached onto a porous support material. The porous support material may include at least one exposed surface constructed to absorb a fluid. The test spot may change color when in contact with THC, e.g., if the salt used is that of Formula (IB), the spot may change from tan or yellow to a bright red color. A user may use the test strip by applying an amount of sample onto the porous support material including the THC sensing gel. If THC is present at a concentration above a detection limit, test spot may exhibit a detectable color change. The color change may be detected by user (e.g., by a naked eye), or by using an instrument, e.g., a UV/VIS reader or colorimeter. The test strip may be utilized, for example, by law enforcement to detect THC in a sample of interest.

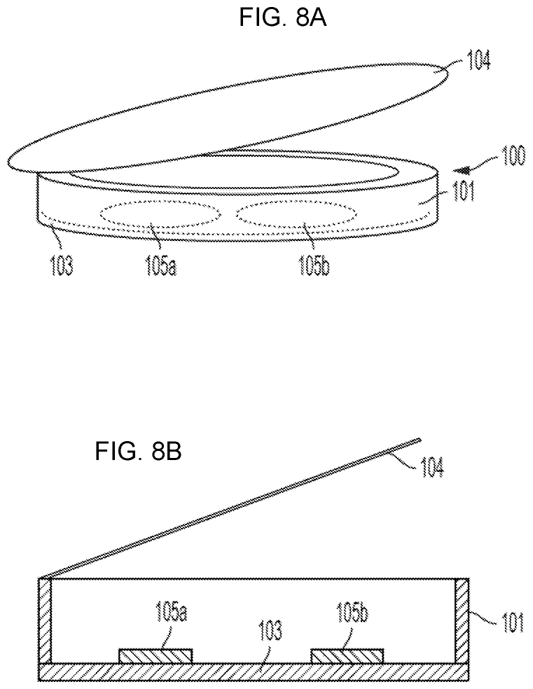

In FIG. 8A, a diagram shows a test device (e.g., a test strip) 100 according to an example embodiment. The test device 100 includes an enclosure 101 in which one or more test spots 105a, 105b, are placed onto a substrate 103. Generally, at least the substrate material 103 is transparent at a wavelength of interest, e.g., in the visible light spectrum. The enclosure 101 is covered prior to use by a removable seal 104. Both the enclosure 101 and seal 104 are generally impermeable to gases and liquids, e.g., air, water, so as to protect the support material and sensing chemicals. FIG. 8B shows a cross-section the test strip.

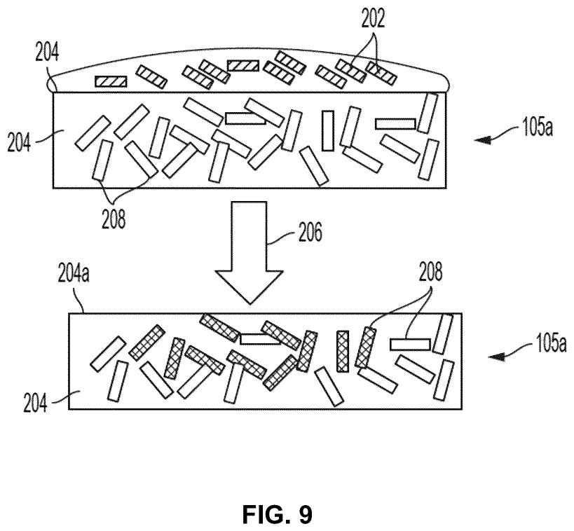

In FIG. 9, a diagram shows a section of the porous support material incorporating sensing chemicals, 105a. In the top of the figure, a sample of fluid 200 (e.g., saliva) is placed on the top surface 204a of the porous support material 204. The sample 200 has a concentrated amount of a drug 202 that is subject to testing. As indicated by the arrow 206, the sample fluid 200 is absorbed into the support material 204 where it contacts at least one of the sensing chemicals 208. As indicated by the shaded sensing chemicals 208, the chemical reacts with the drug 202 and undergoes a color change. The amount of the color change is indicative of a concentration of the drug 202 in the test fluid 200.

The detection limit of the test strip may be adjusted to a certain degree by the selection of the diazonium salt and the amount of THC sensing gel used in the test spots. Test strips with different detection limits may be desirable in different contexts (e.g., law enforcement setting, healthcare setting, at schools and other educational institutions, at the workplace, delivery workers, transportation workers, operators of heavy machinery, government, etc.) In some embodiments, the test strip can detect concentrations of THC of 100 ng/mL or greater, 75 ng/mL or greater, 50 ng/mL or greater, 25 ng/mL, or as low as 10 ng/mL.

The colorimetric test strip described herein may present multiple advantages compared to other THC detection methods currently available, such as immunoassays, multi-solution colorimetric test kits, or mass-spectrometry based assays. The test strip of the present disclosure may be cheaper to produce, have a faster reaction time, and/or be more portable than other THC detection methods. In some embodiments, the test strip may yield more reproducible results and lower rates of false negatives than comparable THC detection rapid tests.

In some embodiments, the diazonium salt in the THC sensing gel is covalently attached to the polymerized monomers and shows less leaching during testing than when the diazonium salt is non-covalently dispersed throughout the polymerized monomers.

In some embodiments, the diazonium salt in the THC sensing gel is covalently attached to the polymerized monomers and has greater stability than when the diazonium salt is non-covalently dispersed throughout the polymerized monomers.

The test strip may be used by applying a sample onto the THC sensing gel (e.g., test spots disposed on or in a porous support). The sample may be a fluid sample. The fluid sample may be absorbed by the porous support. In some embodiments, the fluid absorbed by the test strip is a sample dissolved in a solvent. The sample may be or include at least one of blood, urine, plasma, saliva, or a combination thereof. The test strip may be configured to absorb fluid into the THC sensing gel. In some embodiments, the solvent is a buffer with a pH of 7 or higher. In some of these embodiments, the solvent is organic. The solvent may be aqueous. In some of these embodiments, the solvent is chloroform, acetone, dichloromethane, or methanol. The fluid absorbed by the test strip may be a gas. The fluid absorbed by the test strip may be a droplet suspension, such as a suspension or respiratory droplets.

The test strip may be provided as part of a kit. In some embodiments, a kit comprises a test strip. In some embodiments, the test strip may comprise more than one test spot. There may be more than one test strip included in a kit. In some embodiments, a kit includes at least one test strip and instructions for use. In some embodiments, a kit includes a device to spectrophotometrically analyze the test strip.

In some embodiments, a kit additionally comprises a container with buffer. The buffer may be aqueous. The buffer may be organic. The buffer may additionally comprise a preservative. The buffer may need to be diluted before use with the kit. Additional components may need to be added to the buffer before use. In some embodiments, the kit may include less than 1.0 mL of buffer. The kit may include 1.0-2.0 mL of buffer. The kit may include 1.0 to 5.0 mL of buffer.

The buffer may be provided in a container. The buffer container may be resealable to contain the sample for extended periods of time. The buffer container may be configured to apply sample in buffer to the test strip. The kit may comprise more than one buffer container. The kit may comprise one buffer container for each included test strip.

In some embodiments, a kit may comprise an apparatus for sample collection. In some embodiments, a kit may comprise a swab for sample collection. In some embodiments, a kit may comprise a container for sample collection. The container may be suitable for collection of urine, blood, saliva, plasma, or a combination thereof. In some embodiments, a kit may comprise a blood collection device comprising a needle. In some embodiments, a kit may comprise a syringe for sample collection. The kit may comprise one sample collection apparatus for each included test strip.

In some embodiments, a kit includes a component for sample preparation before application to a test strip. A sample preparation component may be a filter, a syringe, a column, or a lysis device. A sample preparation component may remove physical debris from a sample. A sample preparation component may lyse cells from a sample. A sample preparation component may remove a subset of molecules from a sample. In some embodiments, the sample does not need to be diluted in buffer before application to the test strip.

In some embodiments, a kit comprises a dropper to apply the sample to the test strip. The dropper may be part of the buffer container. The dropper may be a pipette. The dropper may be configured to deliver a precise volume.

The kit may include instructional materials. Instructions for use of a kit to test a sample generally comprise obtaining a sample, optionally preparing the sample for testing, applying the prepared sample to the test strip, and interpreting the results.

According to an embodiment, a method for making a THC sensing gel includes preparing a reaction mixture including aromatic amines, polymerizable moieties, and a solvent. The method next includes polymerizing the reaction mixture by addition of a radical initiator to produce a polymer with the aromatic amines dispersed within the polymer. The method then includes reacting the aromatic amines to yield a composition of a diazonium salt and polymerized monomers.

According to an embodiment, the reaction mixture contains one or more polymerizable monomers and one or more aromatic amines. In some of these embodiments, the polymerizable monomers are mono-, di-, or multi-functional monomers. The polymerizable monomers may include vinyl monomers, acrylate monomers, methacrylate monomers, or a combination thereof. In some embodiments, the polymerizable monomers include allyl phenyl ether and poly(ethylene glycol) diacrylate Mₙ 700. Once polymerized, the polymerizable monomers are capable of forming a gel. Preferably, once polymerized, the polymerizable monomers form a gel.

In some embodiments, the reaction mixture may include only one type of polymerizable monomer. In some embodiments, the reaction mixture may include a first type of polymerizable monomer and a second type of polymerizable monomer. The first and second may be present in reaction mixture at molar ratio of between 0.001:1 and 1:1. The ratio of one monomer to another is not particularly limited, and should be understood by one skilled in the art to be selected to best fit a given circumstance. The reaction mixture may include a molar ratio of between 0.00001:1 to 0.5:1 of initiator relative to total reaction components. The reaction mixture may include a molar ratio of 0.00001:1 to 0.5:1 of diazonium salt relative to total components. In some embodiments, the reaction mixture may include up to 10%, up to 20% or up to 80% w/w solid reaction components.

In some embodiments, the polymerization reaction proceeds for between one and two hours. The polymerization reaction may proceed for as little as five minutes, or as long as 48 hours. The reaction may be scaled up to produce several thousand kilograms of polymer. The reaction may also be scaled down to produce as little as one gram of polymer. In some embodiments, when an amino aryloxyl monomer is included in the reaction mixture, it may comprise up to 1%, up to 5%, up to 10%, or up to 15% of the monomers by weight.

In some embodiments, the solvent used in the reaction mixture may be dichloromethane, acetonitrile, acetone, or a combination thereof.

In some embodiments, the radical initiator is an azo compound such as azobis(4-methoxy-2.4-dimethyl valeronitrile), or 2,2′-azobis(isobutyronitrile, AIBN). In some embodiments, the radical initiator is an organic peroxide such as benzoyl peroxide (BPO), di-tert-butyl peroxide, 2,2′-azobis[2-(2-imidazolin-2-yl)-propane] dihydrochloride, or cumene hydroperoxide.

In some embodiments, the reaction mixture includes one or more of the amino aryloxyl monomers described above (Formula II, Formula IIA, Formula IIA.1, Formula IIA.2, Formula IIB, Formula IIC, Formula IID, and Formula IIE). If an amino aryloxyl monomer is included in the reaction mixture, additional diazonium salt precursors may be included as well. When amino aryloxyl monomers are included in the reaction mixture, polymerized, and subjected to diazonium-activating conditions after polymerization, the resulting diazonium group may be covalently attached to the polymeric structure. This may confer beneficial properties to the sensing gel. Potential beneficial properties include improved thermal stability and decreased leaching during testing.

In some embodiments, the aromatic amines may be activated by addition of sodium nitrite (NaNO₂) and hydrochloric acid (HCl) to yield a diazonium salt. In some embodiments, the aromatic amines may be activated by addition of sodium nitrate (NaNO₂) and an alternate acid/salt solution, such as sulfuric acid, phosphoric acid, picric acid, p-toluene sulfonic acid, carboxylic acids, thiocyanic acid. In some embodiments, the diazonium salt is stabilized by addition of an alternate counterion. While the diazonium salt sometimes has a halide counterion immediately after activation, a larger counterion may be substituted to improve stability of the diazonium salt. In some of these embodiments, the alternate counterion is thiocyanate, picrate, carboxylate, tosylate, or o-benzenedisulfonimide. In some embodiments, the counterion may be exchanged by incubation with excess of the counterion-containing salt in the polymeric gel at a low temperature.

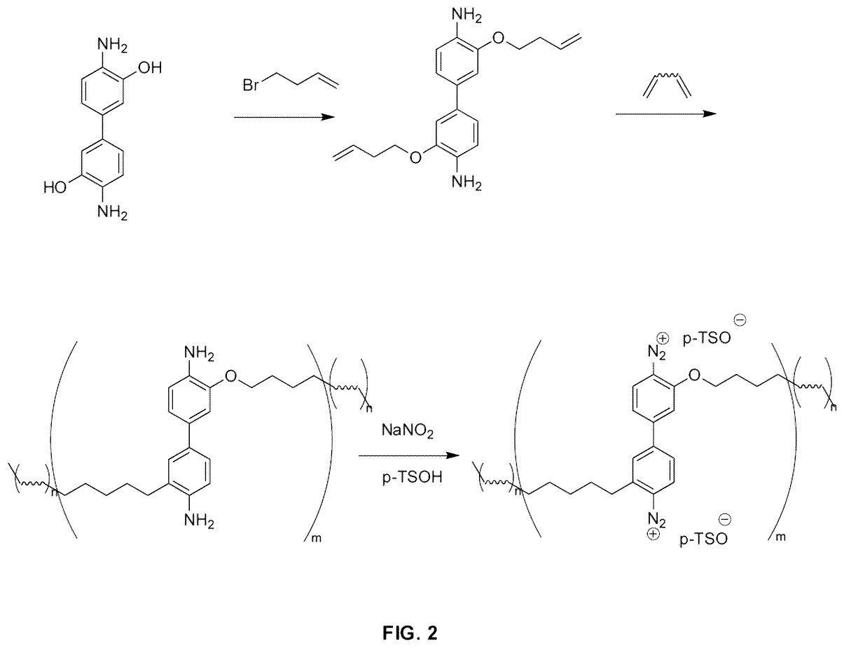

According to an embodiment, a method for making the amino aryloxyl monomer described above includes reacting an aromatic amino phenol and monomer precursor in a solvent to yield an amino aryloxyl monomer. This reaction is shown in step (1) of FIG. 2.

In some embodiments, only a portion of the aromatic amino phenols may be reacted with the monomer precursor. Multiple additions of the monomer may also occur if the provided aromatic amino phenol has multiple suitable alcohol moieties. In these embodiments, each produced monomer may be suitable for subsequent polymerization.

Commercially available diazonium salts are typically not suitable for reaction with a monomer precursor. Aromatic amino phenols suitable for reaction with a monomer precursor may be prepared by obtaining a modified diazonium salt in which the diazonium moiety is replaced with an amino group and which includes a suitably reactive alcohol moiety.

For example, Formula IIIA shows the structure of Fast Blue B salt (o-dianisidine bis(diazotized) zinc double salt), a diazonium salt not suitable for reaction with a monomer precursor. Formula IIIB shows 3-3′-dihydroxybenzidine, the aromatic amino phenol suitable for reaction with monomer precursor. After a monomer precursor has been reacted with the alcohol moieties, 3-3′-dihydroxybenzidine could be further reacted to form diazonium salt from the amino group.

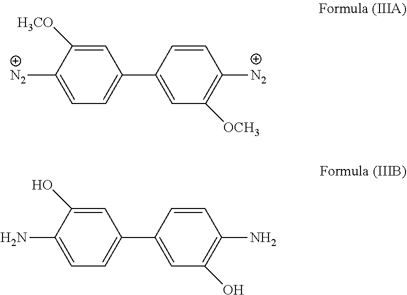

Diazonium salts that may be suitable for modification and reaction with a monomer precursor include Fast Blue B salt (o-dianisidine bis(diazotized) zinc double salt), Fast Blue BB Salt hemi(zinc chloride) (4-benzoylamino-2,5-diethoxybenzenediazonium chloride hemi(zinc chloride) salt), Fast Corinth V (2-methoxy-5-methyl-4-(4-methyl-2-nitro phenyl)-azobenzene-diazonium), Fast Red B (2-methoxy-4-nitro benzenediazonium salt), Fast Bordeaux GP salt (4-methoxy-2-nitro benzenediazonium chloride hemi(zinc chloride) salt), Fast Blue RR salt (4-benzoylamino-2,5-dimethoxybenzenediazonium chloride hemi(zinc chloride) salt), Fast Garnet GBC sulfate salt (4,2-methyl-4-([2-methylphenyl]azo)benzenediazonium salt), and Fast Red Violet LB Salt (5-chloro-4-benzamido-2-methylbenzenediazonium chloride hemi(zinc chloride) salt).

In some embodiments, this method may include further recovering and/or purifying the amino aryloxyl monomer using a chromatography column. Any suitable chromatography column may be used, such as a column including silica or neutral alumina. Neutral alumina may prevent or reduce degradation of the amino aryloxyl monomer during chromatography. Purification of the amino aryloxyl monomer may also be performed by selective precipitation with a mixture of solvents, recrystallization from a solvent, and extraction of the crude product with a suitable solvent that selectively dissolves the amino aryloxyl monomer.

In some embodiments, the monomer precursor used in preparation of the amino aryloxyl monomer may be selected from an alkenyl halide, acryloyl chloride, or methacryloyl chloride. In some of these embodiments, the alkenyl halide may be selected from bromobutene, bromopropene, bromopentene, chlorobutene, chloropropene, chloropentene, iodobutene, iodopropane, iodopentene, or a combination thereof.

### Salt Leaching Test

Diazonium salt may leach from a gel upon addition of a solvent. To test the level of leaching, a piece of test gel was placed in a vial containing solvent such as methanol. THC and a small amount of NaOH were added to the vial and incubated for several minutes. The diazonium salt turned red upon reaction with THC. Leaching was visually assessed by looking at the color of the gel and the solvent. If significant leaching had occurred, the solvent would appear red. Absorbance spectroscopy was used to quantify the level of diazonium salt that had leached into the solvent.

### Thermal Stability Test

Unstable diazonium salts degrade over time and will turn red when degraded. Stability of a diazonium salt in a gel may be assayed by storing a test gel at room temperature or 4° C. over time. The level of diazonium salt degradation may be visually assayed by observing the color of the gel at a given timepoint. Absorbance spectroscopy may be used to quantify the level of degradation.

### EXEMPLARY EMBODIMENTS

The following is a list of exemplary embodiments according to the present disclosure.

Embodiment 1 is a composition for detecting THC comprising polymerized monomers and a diazonium salt,
(i) wherein the diazonium salt is covalently attached to the polymerized monomers; or
(ii) wherein the diazonium salt is non-covalently dispersed throughout the polymerized monomers.
   Embodiment 2 is the composition of embodiment 1, comprising more than one type of polymerized monomer.
   Embodiment 3 is the composition of embodiment 1 or 2, wherein the polymerized monomers comprise mono-, di-, or multi-functional monomers comprising vinyl monomers, acrylate monomers, methacrylate monomers, or a combination thereof. Preferably, the polymerized monomers are difunctional acrylate monomers.
   Embodiment 4 is the composition of any one of embodiments 1 to 3, wherein the diazonium salt is stabilized by addition of an alternate counterion, wherein the alternate counterion is not chloride ion.
   Embodiment 5 is the composition of embodiment 4, wherein the counterion comprises thiocyanate, picrate, carboxylate, tosylate, or o-benzenedisulfonimide, preferably the counterion comprises thiocyanate.
   Embodiment 6 is the composition of any one of embodiments 1 to 5, wherein the diazonium salt is non-covalently dispersed throughout the polymerized monomers, wherein the diazonium salt comprises a compound of Formula (I):

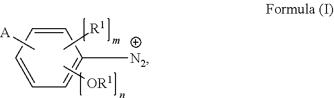

wherein A is an aromatic or nitrogen-containing group, or wherein A is absent, optionally wherein A is an anisole ring substituted with a diazonium group. A may also optionally be NO₂, aniline, or a benzamide group.
Each R¹ is independently alkyl, ether, alcohol, or aromatic, optionally wherein R¹ is a methyl group,
wherein n is 1 or 2, optionally wherein n is 1
wherein each R² is independently —H, -halogen, or alkyl, optionally wherein R² is —H, and
wherein m is 2 or 3, optionally wherein n is 2.
   Embodiment 7 is the diazonium salt of embodiment 6 wherein A is NO₂, a phenyl group attached via nitrogen, or a disubstituted phenyl group.
   Embodiment 8 is the composition of any one of embodiments 1 to 7, wherein the diazonium salt is non-covalently dispersed throughout the polymerized monomers, wherein the diazonium salt comprises a compound of Formula (IA):

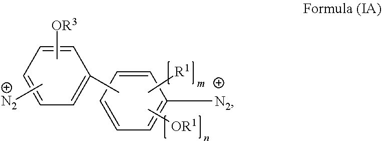

wherein each R¹ is independently —H or alkyl,
wherein n is 1 or 2,
wherein each R² is independently —H, -halogen, or alkyl, optionally wherein R² is —H,
wherein m is 2 or 3, optionally wherein m is 2, and
wherein each R³ is independently —H, alkyl, or ether, optionally wherein R³ is methyl.
   Embodiment 9 is the composition of any one of embodiments 1 to 8, wherein the diazonium salt is non-covalently dispersed throughout the polymerized monomers, wherein the diazonium salt comprises a compound of Formula (IB):

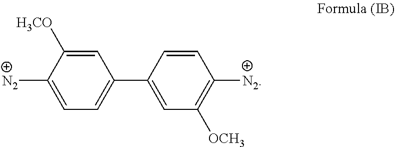

Embodiment 10 is a molecule comprising an amino aryloxyl monomer comprising a polymerizable double bond. The amino aryloxyl monomer may optionally comprise more than one polymerizable double bond. Preferably the molecule is suitable for use in preparing a polymeric sensing gel for detection of THC. The molecule may be copolymerized with other monomers to prepare the polymeric sensing gel.
Embodiment 11 is the molecule of any one of embodiments 1 to 10, wherein the amino aryloxyl monomer comprises a compound of Formula (II):

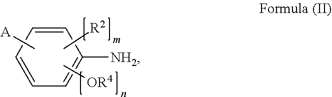

wherein is an aromatic or nitrogen-containing group, or wherein A is absent,
wherein each R⁴ is independently selected from alkyl, -vinyl, acrylate, or methacrylate, optionally wherein R⁴ is a three carbon alkyl chain ending in a vinyl group.
wherein n is 1 or 2,
wherein each R² is independently —H, -halogen, or alkyl,
wherein m is 2 or 3.
   Embodiment 12 is the molecule of embodiment 10, wherein the amino aryloxyl monomer comprises a compound of Formula (IIA):

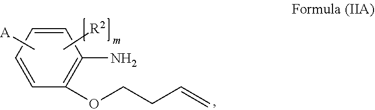

wherein A is an aromatic or nitrogen-containing group, or wherein A is absent, preferably
   wherein A is an allyl phenyl ether substituted with an amino group,
wherein each R² is independently —H, -halogen, or alkyl, preferably wherein R² is —H
wherein m is 2 or 3, preferably wherein m is 3.
   Embodiment 13 is the molecule of embodiments 11-12 wherein A is NO₂, a phenyl group attached via nitrogen, or a disubstituted phenyl group. A may also optionally be NO₂, aniline, or a benzamide group.
   Embodiment 14 is the molecule of embodiment 10, wherein the amino aryloxyl monomer comprises a compound of Formula (IIB):

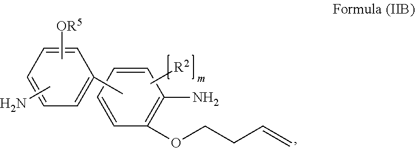

wherein each R⁵ is independently alkyl, vinyl, acrylate, or methacrylate, optionally wherein R⁵ is an alkyl vinyl group,
wherein each R² is independently —H, -halogen, or alkyl, optionally wherein R² is —H,
wherein m is 2 or 3, optionally wherein m is 2.
   Embodiment 15 is the molecule of embodiment 10, wherein the amino aryloxyl monomer comprises a compound of Formula (IIC):

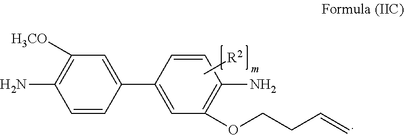

wherein each R² is independently —H, -halogen, or alkyl.
wherein m is 2 or 3.
   Embodiment 16 is the molecule of embodiment 10, wherein the amino aryloxyl monomer comprises a compound of Formula (IID) or Formula (IIE):

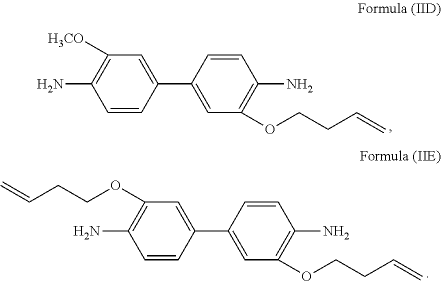

Embodiment 17 is the composition of embodiment 1, wherein the diazonium salt is covalently attached to the polymerized monomers,
wherein the diazonium salt comprises at least one of the molecules of embodiments 10-16, and
wherein the amino group has been replaced with a diazonium group, optionally by reaction with sodium nitrite and hydrochloric acid.
   Embodiment 18 is a test strip comprising:
a substantially transparent substrate; and
one or more test spots comprising a composition for detecting THC attached onto a porous support material, the composition comprising polymerized monomers and a diazonium salt, (i) wherein the diazonium salt is covalently attached to the polymerized monomers; or (ii) wherein the diazonium salt is non-covalently dispersed throughout the polymerized monomers,
wherein the porous support material has at least one exposed surface constructed to absorb a fluid.
   Embodiment 19 is the test strip of embodiment 18, wherein the test strip comprises a single (1) test spot.
   Embodiment 20 is the test strip of embodiment 18 or 19, wherein the diazonium salt is covalently attached to the polymerized monomers and is formed from the molecule of one of Embodiments 11 to 17.
   Embodiment 21 is the test strip of any one of embodiments 18 to 20, wherein the fluid comprises sample dissolved in a solvent, wherein optionally the sample comprises at least one of blood, urine, plasma, saliva, or a combination thereof.
   Embodiment 22 is the solvent of embodiment 21, wherein the solvent comprises a buffer with a pH of 7 or higher.
   Embodiment 23 is the test strip of embodiment 21, wherein the solvent is organic.
   Embodiment 24 is the test strip of embodiment 23, wherein the solvent comprises chloroform, acetone, dichloromethane, dimethylformamide, ethanol, ethylene glycol, diethylene glycol, acetonitrile or methanol.
   Embodiment 25 is a method for making the composition of embodiment 1, the method comprising:
preparing a reaction mixture comprising aromatic amines, polymerizable monomers, and solvent;
polymerizing the reaction mixture by addition of a radical initiator to produce a polymer with the aromatic amines dispersed within the polymer; and
reacting the aromatic amines to yield a composition comprising a diazonium salt and polymerized monomers.
   Embodiment 26 is the method of embodiment 25, wherein the reaction mixture comprises one or more types of polymerizable monomers, one or more types of aromatic amines, or both.
   Embodiment 27 is the method of embodiment 25 or 26, wherein the polymerizable monomers comprise mono-, di-, or multi-functional monomers comprising vinyl monomers, acrylate monomers, methacrylate monomers, or a combination thereof.
   Embodiment 28 is the method of any one of embodiments 25 to 27, wherein the reaction mixture comprises the molecule of embodiment 10.
   Embodiment 29 is the method of any one of embodiments 25 to 28, wherein the diazonium salt is stabilized by addition of an alternate counterion, wherein the alternate counterion is not chloride ion.
   Embodiment 30 is the method of embodiment 29, wherein the counterion comprises thiocyanate, picrate, carboxylate, tosylate, or o-benzenedisulfonimide.
   Embodiment 31 is the method of any one of embodiments 25 to 30, wherein the solvent comprises dichloromethane, acetonitrile, acetone, methanol, ethanol or a combination thereof.
   Embodiment 32 is the method of any one of embodiments 25 to 31, wherein the aromatic amines are activated by addition of sodium nitrite (NaNO₂) and hydrochloric acid (HCl).
   Embodiment 33 is a method for making the amino aryloxyl monomer of embodiment 10, the method comprising:
reacting an aromatic amino phenol and monomer precursor in a solvent to yield an amino aryloxyl monomer.
   Embodiment 34 is the method of embodiment 33, further comprising recovering the amino aryloxyl monomer using a neutral alumina chromatography column.
   Embodiment 35 is the method of embodiment 33 or 34, wherein the monomer precursor is selected from an alkenyl halide, acryloyl chloride, or methacryloyl chloride.
   Embodiment 36 is the method of embodiment 35, wherein the alkenyl halide is selected from bromobutene, bromopropene, bromopentene, chlorobutene, chloropropene, chloropentene, iodobutene, iodopropane, iodopentene, or a combination thereof.
   Embodiment 37 is a composition for detecting THC comprising polymerized monomers and a diazonium salt, wherein the diazonium salt is covalently attached to the polymerized monomers.
   Embodiment 38 is the composition of embodiment 37, wherein the polymerized monomers form a gel.
   Embodiment 39 is the composition of embodiment 37, comprising more than one type of polymerized monomer.
   Embodiment 40 is the composition of embodiments 37, wherein the polymerized monomers comprise mono-, di-, or multi-functional monomers comprising vinyl monomers, acrylate monomers, methacrylate monomers, or a combination thereof.
   Embodiment 41 is the composition of embodiment 37, wherein the diazonium salt is stabilized by addition of an alternate counterion.
   Embodiment 42 is the composition of embodiment 41, wherein the counterion comprises thiocyanate, picrate, carboxylate, tosylate, or o-benzenedisulfonimide.
   Embodiment 43 is the composition of embodiment 41, wherein the diazonium salt comprises an amino aryloxyl monomer of Formula (II):

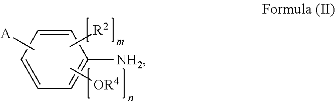

wherein is an aromatic or nitrogen-containing group, or wherein A is absent,
wherein each R⁴ is independently selected from alkyl, -vinyl, acrylate, or methacrylate, optionally a three carbon alkyl chain ending in a vinyl group
wherein n is 1 or 2,
wherein each R² is independently —H, -halogen, or alkyl,
wherein m is 2 or 3,
wherein at least one of the R⁴ is covalently attached to the polymerized monomers.
   Embodiment 44 is the composition of embodiment 43, wherein the amino aryloxyl monomer comprises a compound of Formula (IIA):

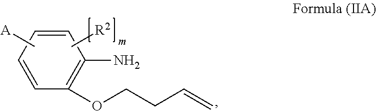

wherein A is an aromatic or nitrogen-containing group, or wherein A is absent, preferably wherein A is an allyl phenyl ether substituted with an amino group,
wherein each R² is independently —H, -halogen, or alkyl, preferably wherein R² is —H
wherein m is 2 or 3, preferably wherein m is 3.
   Embodiment 45 is the composition of embodiment 43 or 44, wherein A is NO₂, a phenyl group attached via nitrogen, or a disubstituted phenyl group.
   Embodiment 46 is the composition of embodiment 43, wherein the amino aryloxyl monomer comprises a compound of Formula (IIB):

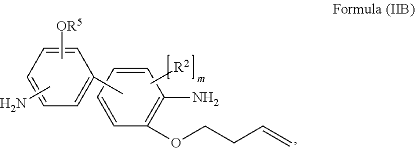

wherein each R⁵ is independently alkyl, vinyl, acrylate, or methacrylate,
wherein each R² is independently —H, -halogen, or alkyl, preferably wherein R² is —H
wherein m is 2 or 3, preferably wherein m is 3.
   Embodiment 47 is the composition of embodiment 43, wherein the amino aryloxyl monomer comprises a compound of Formula (IIC):

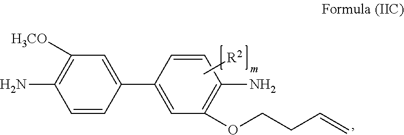

wherein each R² is independently —H, -halogen, or alkyl,
wherein m is 2 or 3.
   Embodiment 48 is the composition of embodiments 37 to 43, wherein the amino aryloxyl monomer comprises a compound of Formula (IID) or Formula (IIE):

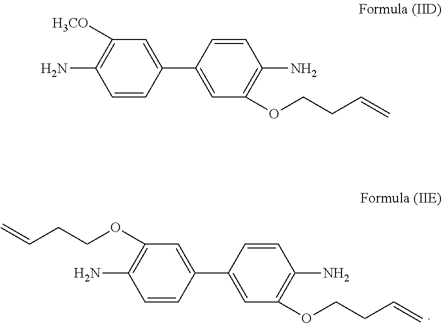

Embodiment 49 is a composition for detecting THC comprising polymerized monomers and a diazonium salt, wherein the diazonium salt is non-covalently dispersed throughout the polymerized monomers.
Embodiment 50 is the compositions of embodiments 49, wherein the polymerized monomers form a gel.
Embodiment 51 is the composition of embodiment 49, comprising more than one type of polymerized monomer.
Embodiment 52 is the composition of embodiments 49, wherein the polymerized monomers comprise mono-, di-, or multi-functional monomers comprising vinyl monomers, acrylate monomers, methacrylate monomers, or a combination thereof.
Embodiment 53 is the composition of embodiment 49, wherein the diazonium salt is stabilized by addition of an alternate counterion.
Embodiment 54 is the composition of embodiment 53, wherein the counterion comprises thiocyanate, picrate, carboxylate, tosylate, or o-benzenedisulfonimide.
Embodiment 55 is the composition of embodiment 49, wherein the diazonium salt is non-covalently dispersed throughout the polymerized monomers, wherein the diazonium salt comprises a compound of Formula (I):

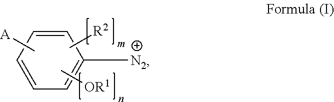

wherein A is an aromatic or nitrogen-containing group, or wherein A is absent,
wherein each R¹ is independently alkyl, ether, alcohol, or aromatic,
wherein n is 1 or 2,
wherein each R² is independently —H, -halogen, or alkyl, and
wherein m is 2 or 3.
   Embodiment 56 is the diazonium salt of embodiment 55 wherein A is NO₂, a phenyl group attached via nitrogen, or a disubstituted phenyl group.
   Embodiment 57 is the composition of embodiment 49, wherein the diazonium salt is non-covalently dispersed throughout the polymerized monomers, wherein the diazonium salt comprises a compound of Formula (IA):

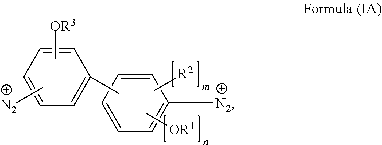

wherein each R¹ is independently —H or alkyl,
wherein n is 1 or 2,
wherein each R² is independently —H, -halogen, or alkyl,
wherein m is 2 or 3, and
wherein each R³ is independently —H, alkyl, or ether.
   Embodiment 58 is the compositions of embodiments 48 or 49, wherein the diazonium salt is non-covalently dispersed throughout the polymerized monomers, wherein the diazonium salt comprises a compound of Formula (IB):

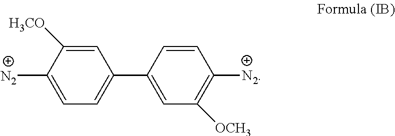

Embodiment 59 is a kit for testing for presence of THC in a sample, the kit comprising:
a test device (e.g., a test strip) comprising:
   a substantially transparent substrate; and
   one or more test spots comprising a composition for detecting THC attached onto a porous support material, the composition comprising polymerized monomers and a diazonium salt, (i) wherein the diazonium salt is covalently attached to the polymerized monomers; or (ii) wherein the diazonium salt is non-covalently dispersed throughout the polymerized monomers; and
instruction materials for using the test strip.
   Embodiment 60 is the kit of embodiment 59, wherein the instruction materials include instructions to obtain a sample, optionally to prepare the sample for testing, to apply the prepared sample to the test strip, and to interpret results.
   Embodiment 61 is the kit of embodiment 60, wherein preparing the sample for testing comprises mixing the sample with a buffer.
   Embodiment 62 is the kit of embodiment 60, wherein the interpreting of the results comprises observing a color change on the test device.
   Embodiment 63 is the kit of any one of embodiments 59 to 62 further comprising a buffer, optionally wherein the buffer is provided in a container. The buffer may be aqueous. The buffer may be organic. The buffer may additionally comprise a preservative. The buffer may be diluted before use with the kit. In some embodiments, the kit may include less than 1.0 mL of buffer. The kit may include 1.0-2.0 mL of buffer. The kit may include 1.0 to 5.0 mL of buffer.
   Embodiment 64 is the kit of any one of embodiments 59 to 63 further comprising an apparatus for sample collection, optionally wherein the apparatus comprises a swab, alternatively optionally wherein the apparatus comprises a container.
   Embodiment 65 is the kit of any one of embodiments 59 to 64 further comprising a sample applicator, such as a dropper or pipette.
   Embodiment 66 is the kit of any one of embodiments 59 to 65 further comprising a sample preparation component, optionally wherein the sample preparation component comprises a filter, a syringe, a column, a lysis device, or a combination thereof.

### EXAMPLES

### Example 1

In Example 1, a polymer incorporating disperse diazonium salt was prepared. A polymer may be prepared according to the following method, using the compounds shown in TABLE 1.

TABLE 1

| | Component | Amount (g) | Obtainable from |
|---|---|---|---|
| 1 | Allyl phenyl ether | 0.75 | AK Scientific, CAS |
| | | | Number:, 1746-13-0 |
| 2 | Poly(ethylene glycol) | 0.96 | Sigma, CAS Number: |
| | diacrylate, Mₙ 700 | | 26570-48-9 |
| 3 | O-dianisidine | | TCI America, CAS |
| | | | Number: 20325-40-0 |
| 4 | Acetonitrile | | Sigma, CAS Number: |
| | | | 75-05-8 |
| 5 | 2,2′-Azobis(4-methoxy-2,4- | 0.120 | Fuji Film, Wako |
| | dimethyl valeronitrile | | chemicals, CAS |
| | | | Number: 15545-97-8 |
| 6 | Hydrochloric acid | 0.93 | Sigma, CAS Number: |
| | | | 7647-01-0 |
| 7 | Sodium nitrite | 0.58 | Sigma, CAS Number: |
| | | | 7632-00-0 |
| 8 | Hydrochloric acid | 1.05 | Sigma, CAS Number: |
| | | | 7647-01-0 |
| 10 | Sodium thiocyanate | 0.03567 | Sigma, CAS Number: |
| | | | 540-72-7 |

In a first step, the free radical of a polymer network based on allyl phenyl ether and poly(ethylene glycol) diacrylate was achieved assuming 1:1 mole ratio (0.0056 moles). O-dianisidine (precursor of Fast Blue B salt) was added simultaneously during the polymerization in the acetonitrile (4 mg/mL⁻¹, 6.5 g) as the common solvent. 120 mg of the polymerization initiator 2,2′-azobis(4-methoxy-2.4-dimethyl valeronitrile) (V-70) was added and polymerization occurred under inert atmosphere at 37° C. to yield the amine precursor incorporated polymer network for one hour. The network was subsequently swollen in 50 mL water and 775 μL concentrated hydrochloric acid at 100° C. for 1 hour before cooling the system to 15° C.

In a second step, 0.58 g sodium nitrite in 2.50 mL water along with 875 μL HCl were added dropwise to the network and the reaction vessel was allowed to stir for another two hours at 15° C. At this point the chloride salt of Fast Blue B salt was synthesized in the polymer structure. The dark red sample was washed with copious amount of water and acetonitrile, filtered, and protected from light for downstream stabilization by the counterion.

In a third step, the chloride counterion was exchanged for a thiocyanate counterion to stabilize the diazonium salt. The above network was swollen in water for one hour at room temperature. Then, an equivalent amount of dichloromethane with respect to water was added to the flask, and it was cooled down on ice. At this point, sodium thiocyanate was added and the reaction was allowed to stir for another two hours. Finally, the sample was washed with excess of water by centrifugation.

It was observed that the gel produced had a yellow color and that there was no visible leaching during the wash steps. It was concluded that the O-dianisidine had been incorporated into the gel and activated to form the diazonium salt.

### Example 2

In Example 2, a test spot polymer incorporating a modified diamine diol Formula (IVA)-derived monomer Formula (IVB) was prepared.

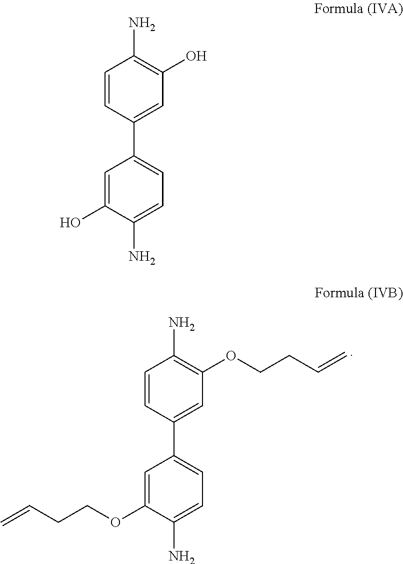 [SMILES: Nc1ccc(-c2ccc(N)c(O)c2)cc1O ; C=CCCOc1cc(-c2ccc(N)c(OCCC=C)c2)ccc1N]

A molecule may be prepared according to TABLE 2.

TABLE 2

| | Component | Amount (g) | Obtainable from |
|---|---|---|---|
| 1 | 4,4′-Diamino-[1,1′- | 5 | AK Scientific, CAS |
| | biphenyl]-3,3′-diol (I) | | Number: 2373-98-0 |
| 2 | Acetone | 180 | Sigma-Aldrich CAS |
| | | | Number 67-64-1 |
| 3 | Acetonitrile | 180 | Sigma-Aldrich, CAS |
| | | | Number: 75-05-8 |
| 4 | 4-Bromo-1-butene | | TCI America ™, CAS |
| | | | Number: 5162-44-7 |
| 5 | Anhydrous potassium | 19 | Sigma-Aldrich, CAS |
| | carbonate | | Number: 497-19-8 |
| 6 | Dichloromethane | 250 | Sigma-Aldrich, CAS |
| | | | Number: 75-09-2 |
| 7 | Sodium sulfate | 30 | Sigma-Aldrich, CAS |
| | | | Number: 7757-82-6 |
| 8 | Hexane | 1000 | Sigma-Aldrich CAS |
| | | | Number: 110-54-3 |
| 9 | Ethyl acetate | 1500 | Sigma-Aldrich CAS |
| | | | 141-78-6 |

In a first step, 4,4′-diamino-[1,1′-biphenyl]-3,3′-diol Formula (IVA) was dissolved in a suitable solvent (tested acetone and acetonitrile), with 2.1 equivalents of 4-bromobutene and 3 equivalents of solid anhydrous sodium carbonate and heated to solvent reflux temperature under flow of nitrogen for 20-48 hours. The mixture was dark-brown at the end of the reaction. Solvent was evaporated, the oil was dissolved in dichloromethane, the liquid phase (containing the reaction product) was filtered and washed 2 times with water. After drying over sodium sulfate, the liquid was filtered, and the solvent was evaporated. The crude product was purified by column chromatography on neutral alumina using hexane/ethyl acetate as eluent.

Depending on solvent used and the reaction time, the reaction mixture contained a small fraction of a tetra-vinyl compound, a tri-vinyl-compound, and the target compound Formula (IVB). Characterization and identification confirmed the chemical structure and purity of the identified compounds (1H-NMR, 13C-NMR and mass spectroscopy).

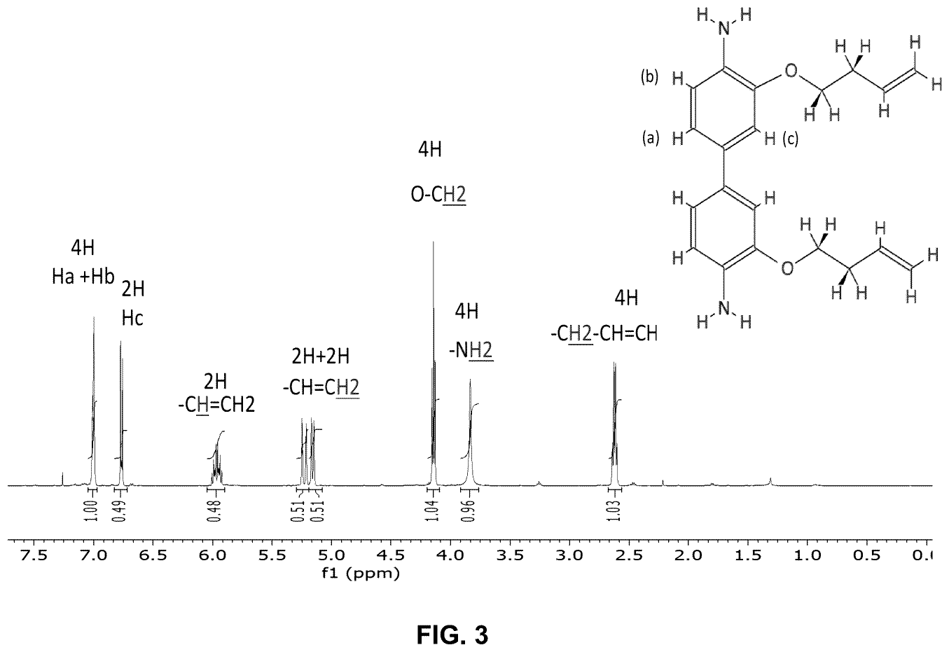

The 1H-NMR and protons assignments for the product Formula (IVB) are shown in FIG. 3.

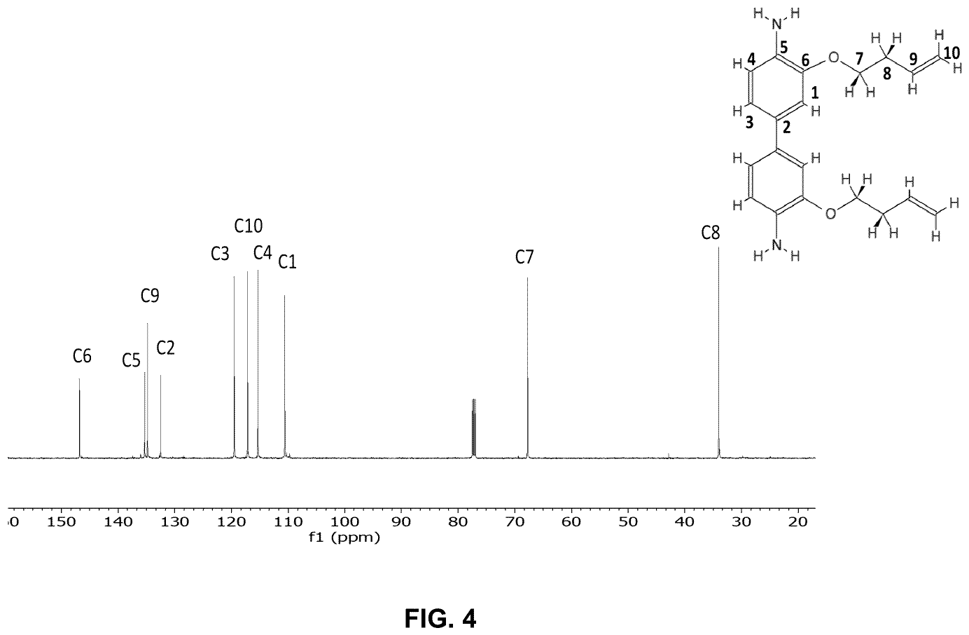

The 13C-NMR confirmed a symmetrical product and allowed identification of all the carbon atoms are shown in FIG. 4.

It was observed that mass spectroscopy (positive Q1) showed an M+1 peak (m/z=325.3) as expected for the target compound Formula (IVB). However, no M+(m/z=324) peak was observed. The mass spectroscopy (negative Q1) shows a peak (m/z=268.9) which can be assigned to the ion corresponding to the loss of —CH2-CH2-CH═CH2 group; FW-55). However, no M+(m/z=324) peak was observed.

It was concluded that a modified diamino monomer of the formula IVB monomer including one, two, three, or four vinyl groups was produced.

### Example 3

In Example 3, a copolymer of acrylate and the modified monomer produced in Example 2 was prepared. A copolymer may be prepared using TABLE 3.

TABLE 3

| | Component | Amount (g) | Obtainable from |
|---|---|---|---|
| 1 | Poly(ethylene glycol) | 0.4026 | Sigma-Aldrich, CAS |
| | diacrylate, Mₙ 700 | | Number: 26570-48-9 |
| 2 | Acetonitrile | 1.62 | Sigma-Aldrich, CAS |
| | | | Number: 75-05-8 |
| 3 | 2,2′-Azobis(4-methoxy-2,4- | 0.03 | Fuji Film, Wako |
| | dimethyl valeronitrile | | Chemicals. CAS |
| | | | Number: 15545-97-8 |
| 4 | p-Toluene sulfonic acid | 0.4 | Sigma, CAS Number: |
| | | | 6192-52-5 |
| 5 | Sodium nitrite | 0.188 | Sigma, CAS Number: |
| | | | 7632-00-0 |

0.4026 g Poly(ethylene glycol) diacrylate (PEG-DA) was dissolved in 1.62 g of Acetonitrile containing 4 mg·mL⁻¹ of Formula IVB. V-70 (30 mg) was added as the initiator. The reaction was carried out under inert atmosphere for 1 hour at 60° C. and a solid puck was extracted as the final product. The samples were washed thrice with acetonitrile and the solvent was exchanged with water with consecutive washing with water thrice. The sample was yellow-tinted and the follow-up washing steps did not yield any significant color changes implying the covalent incorporation of Formula IVB.

In a second step, 0.40 g p-Toluene sulfonic acid in 2.00 mL water was added to the polymer reaction. The sample was stirred for 90 minutes at room temperature. Then, 0.188 g of sodium nitrite in 1 mL water was added dropwise to the network and the reaction vessel was allowed to stir for another 5 hours at room temperature. The sample was the kept in the fridge for 16 hours, then was washed twice with water then with methanol, thrice to provide a yellow gel polymer compound. The sample was kept in methanol and in the fridge until use. It was concluded that a modified Fast Blue B salt of the crosslinked monomer of the formula IIIB was produced.

It was observed that the gel produced was yellow tinted. It was concluded that a copolymer of acrylate and the modified diamino monomer shown in Formula IVB was produced.

### Example 4

In Example 4, a test spot containing a covalently polymerized diazonium salt was used to detect THC.

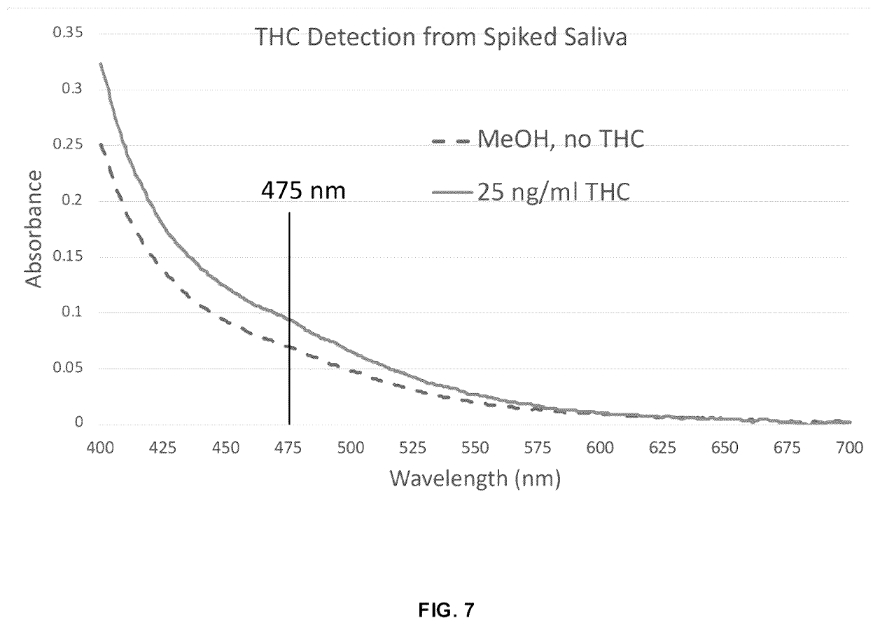

A test spot fabricated as described in Example 3 was used to detect THC (1 mg/mL Sigma) from a spiked saliva test sample in ammonium acetate solution. Representative photographs of a gel before and after testing are shown in FIG. 6. The continuous line in FIG. 7 shows the UV-Vis absorption spectrum of the spiked saliva sample containing 25 ng/mL of THC. The dashed line traces the UV Vis spectrum of the test spot with pure (no THC) saliva. The increase in the intensity of the absorption in the region around 475 nm demonstrates positive THC detection due to the formation of the azo compound upon reaction between the diazonium salt and the THC molecule in gel. It was observed that addition of the spiked saliva solution caused the test spot to have increased absorbance at 475. It was concluded that the test spot was able to detect THC.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A composition for detecting THC comprising polymerized monomers and a diazonium salt,
(i) wherein the diazonium salt is covalently attached to the polymerized monomers; or
(ii) wherein the diazonium salt is non-covalently dispersed throughout the polymerized monomers.

2. The composition of claim 1, comprising more than one type of polymerized monomer.

3. The composition of claim 1, wherein the polymerized monomers comprise mono-, di-, or multi-functional monomers comprising vinyl monomers, acrylate monomers, methacrylate monomers, or a combination thereof.

4. The composition of claim 1, wherein the diazonium salt is stabilized by addition of an alternate counterion.

5. The composition of claim 4, wherein the counterion comprises thiocyanate, picrate, carboxylate, tosylate, or o-benzenedisulfonimide.

6. The composition of claim 1, wherein the diazonium salt is non-covalently dispersed throughout the polymerized monomers, wherein the diazonium salt comprises a compound of Formula (I): 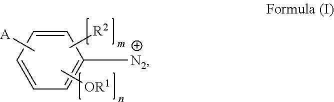
wherein A is an aromatic or nitrogen-containing group, or wherein A is absent,
wherein each R¹ is independently alkyl, ether, alcohol, or aromatic,
wherein n is 1 or 2,
wherein each R² is independently —H, -halogen, or alkyl, and
wherein m is 2 or 3.

7. The diazonium salt of claim 6 wherein A is NO₂, a phenyl group attached via nitrogen, or a disubstituted phenyl group.

8. The composition of claim 1, wherein the diazonium salt is non-covalently dispersed throughout the polymerized monomers, wherein the diazonium salt comprises a compound of Formula (IA): 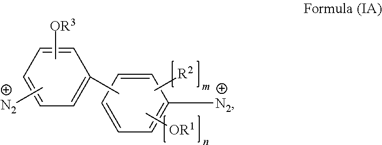
wherein each R¹ is independently —H or alkyl,
wherein n is 1 or 2,
wherein each R² is independently —H, -halogen, or alkyl,
wherein m is 2 or 3, and
wherein each R³ is independently —H, alkyl, or ether.

9. The composition of claim 1, wherein the diazonium salt is non-covalently dispersed throughout the polymerized monomers, wherein the diazonium salt comprises a compound of Formula (IB):

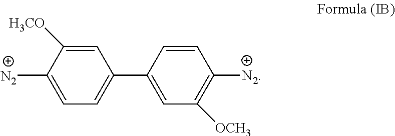

10. A molecule comprising an amino aryloxyl monomer comprising a polymerizable double bond,
wherein the amino aryloxyl monomer comprises a compound of Formula (II): 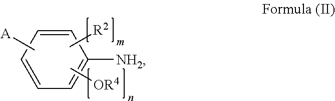
wherein is an aromatic or nitrogen-containing group, or wherein A is absent,
wherein each R⁴ is independently selected from alkyl, -vinyl, acrylate, or methacrylate,
wherein n is 1 or 2,
wherein each R² is independently —H, -halogen, or alkyl,
wherein m is 2 or 3.

11.-17. (canceled)

18. A test strip comprising:
a substantially transparent substrate; and
one or more test spots comprising the composition of claim 1 attached onto a porous support material, wherein the porous support material has at least one exposed surface constructed to absorb a fluid.

19. The test strip of claim 18, wherein the diazonium salt is covalently attached to the polymerized monomers, and wherein the diazonium salt shows less leaching during testing than when the diazonium salt is non-covalently dispersed throughout the polymerized monomers.

20. The test strip of claim 18, wherein the diazonium salt covalently attached to the polymerized monomers, and wherein the diazonium salt has greater thermal stability than when the diazonium salt is non-covalently dispersed throughout the polymerized monomers.

21. The test strip of claim 18, wherein the fluid comprises sample dissolved in a solvent, wherein optionally the sample comprises at least one of blood, urine, plasma, saliva, or a combination thereof.

22. The test strip of claim 21, wherein the solvent comprises a buffer with a pH of 7 or higher.

23.-24. (canceled)

25. A method for making a composition for detecting THC comprising polymerized monomers and a diazonium salt,
(i) wherein the diazonium salt is covalently attached to the polymerized monomers; or
(ii) wherein the diazonium salt is non-covalently dispersed throughout the polymerized monomers, the method comprising:
preparing a reaction mixture comprising aromatic amines, polymerizable moieties, and solvent;
polymerizing the reaction mixture by addition of a radical initiator to produce a polymer with the aromatic amines dispersed within the polymer; and
reacting the aromatic amines to yield a composition comprising a diazonium salt and polymerized monomers.

26. The method of claim 25, wherein the reaction mixture comprises one or more types of polymerizable monomers, one or more types of aromatic amines, or both.

27. The method of claim 25, wherein the polymerizable monomers comprise mono-, di-, or multi-functional monomers comprising vinyl monomers, acrylate monomers, methacrylate monomers, or a combination thereof.

28.-30. (canceled)

31. The method of claim 25, wherein the solvent comprises dichloromethane, acetonitrile, acetone, methanol, ethanol or a combination thereof.

32. The method of claim 25, wherein the aromatic amines are activated by addition of sodium nitrite (NaNO₂) and hydrochloric acid (HCl).

33.-58. (canceled)
